SEQUENCE LISTING (31 sequences)

SEQ ID NO: 1 - protein, 113 aa, Curculigo latifolia
DSVLLSGQTLYAGHSLTSGSYTLTIQNNCNLVKYQHGRQIWASDTDGQGSQCRLTLRSDGNLIIYDDNNMVVWGSDCWGNNGTYALVLQQDGLFVIYGPVLWPLGLNGCRSLN

SEQ ID NO: 2 - protein, 115 aa, Curculigo latifolia
DNVLLSGQTLHADHSLQAGAYTLTIQNKCNLVKYQNGRQIWASNTDRRGSGCRLTLLSDGNLVIYDHNNNDVWGSACWGDNGKYALVLQKDGRFVIYGPVLWSLGPNGCRRVNGG

SEQ ID NO: 3 - DNA, 339 bp, Curculigo latifolia
gacagtgtcctgctctccgggcaaactctgtatgccggccactccctcacgtcgggcagctataccttaaccatacaaaacaactgcaacctggtgaaataccagcacgggaggcagatctgggctagcgacactgacgggcagggctcccaatgccgcctcacattgcggagtgacgggaacctcattatctacgacgacaacaacatggtcgtgtgggggagcgactgctgggggaacaacggcacgtatgctcttgttcttcagcaggatggcctctttgtcatctatggcccggttttgtggccccttggccttaatgggtgccgcagtcttaat

SEQ ID NO: 4 - DNA, 345 bp, Curculigo latifolia
gacaatgtcctgctctccgggcaaactctgcatgccgaccactctctccaggcgggcgcctataccttaaccatacaaaacaagtgcaacctggtgaaataccagaacgggaggcagatctgggctagcaacactgacaggcggggctccggctgccgcctcacattgctgagtgacgggaacctcgttatctacgaccacaacaacaacgacgtgtgggggagcgcctgctggggggacaacggcaagtatgctcttgttcttcagaaggatggcagatttgtcatctatggcccggttttgtggtcccttggccctaatgggtgccgccgtgttaatggtgga

SEQ ID NO: 5 - DNA, 591 bp, Curculigo latifolia
acaatggcggccaagtttcttctcaccattcttgtcacctttgcggccgtcgctagccttggcatggccgacagtgtcctgctctccgggcaaactctgtatgccggccactccctcacgtcgggcagctataccttaaccatacaaaacaactgcaacctggtgaaataccagcacgggaggcagatctgggctagcgacactgacgggcagggctcccaatgccgcctcacattgcggagtgacgggaacctcattatctacgacgacaacaacatggtcgtgtgggggagcgactgctgggggaacaacggcacgtatgctcttgttcttcagcaggatggcctctttgtcatctatggcccggttttgtggccccttggccttaatgggtgccgcagtcttaatggtgaaatcacagttgctaaggattctactgaaccacaacatgaggatattaagatggtgattaataattaatcaagtgagaggattgttatgagaataatgagggaatggaagaccaatctcatgtcggtgtggcctatctcgacctgtttgcagtgcctttgttaaaataacacattgctt

SEQ ID NO: 6 - DNA, 27 bp, Artificial
  Primer 1
cgggatccggacagtgtcctgctctcc

SEQ ID NO: 7 - DNA, 30 bp, Artificial
  Primer 2
ccgctcgagttaattaagactgcggcaccc

SEQ ID NO: 8 - DNA, 23 bp, Artificial
  Primer 3
ggcgccggcatacagagtttgcc

SEQ ID NO: 9 - DNA, 22 bp, Artificial
  Primer 4
tccctcacgtcgggcagctata

SEQ ID NO: 10 - DNA, 25 bp, Artificial
  Primer 5
ggcctggtatttcaccaggttgcag

SEQ ID NO: 11 - DNA, 19 bp, Artificial
  Primer 6
gggaggcagatctgggcta

SEQ ID NO: 12 - DNA, 1166 bp, Curculigo latifolia
cgcaaagacaatggcggccaagtttcttctcaccattcttgtcacctttgcggccgtcgctagccttggcatggccgacaatgtcctgctctccgggcaaactctgcatgccgaccactctctccaggcgggcgcctataccttaaccatacaaaacaagtgcaacctggtgaaataccagaacgggaggcagatctgggctagcaacactgacaggcggggctccggctgccgcctcacattgctgagtgacgggaacctcgttatctacgaccacaacaacaacgacgtgtgggggagcgcctgctggggggacaacggcaagtatgctcttgttcttcagaaggatggcagatttgtcatctatggcccggttttgtggtcccttggccctaatgggtgccgccgtgttaatggtggaatcacagttgctaaggattctactgaaccacaacatgaggatattaagatggtgattaataattaatcaagtgagaggattgttatgagaataatgagtggaatggaagaccaatctcatgtcggtgtggcctatctccacctgtttgcagtgcctttgttaaaataacacattggggaataataaagtgaaactatatagattggttcagcaaattttctgttcagttttcctctcacatgtcaatgtcgattttttgccgcggatcatacatgtgcttggtattctaatcgatagaattatggctcaaatggaggcagggattatgagagtttattcgcatctccgggtcttccaacttacgaattataacaagattcaaggatgcatctgagagccaacttaacgtcttacatcaaaggagctagccgaagtttattcccagagctagaggaagttcgctgccatggttgatagtacaagtagaacgacgcatgtattgcttccaggaatcacttccagcttctcgacacctccagtggccttttcaccaccgaaagcaccaccaatttcagcaccattggtaggtatatttacattaacaataccacagtcactgccatggggtccaatccacttgaaaataacttcaggtctacgagtgaaaatagaactgcttaaacttgcggtacagagttatttatttcaattgcttctttcagagtctggaatttcattacgtaaa

SEQ ID NO: 13 - DNA, 27 bp, Artificial
  Primer 7
cgggatccggacaatgtcctgctctcc

SEQ ID NO: 14 - DNA, 30 bp, Artificial
  Primer 8
ccgctcgagttatccaccattaacacggcg

SEQ ID NO: 15 - DNA, 22 bp, Artificial
  Primer 9
ggccagagtttgcccggagagc

SEQ ID NO: 16 - DNA, 18 bp, Artificial
  Primer 10
gccgaccactctctccag

SEQ ID NO: 17 - DNA, 23 bp, Artificial
  Primer 11
ggcgtcggcagccagagtttgcc

SEQ ID NO: 18 - DNA, 18 bp, Artificial
  Primer 12
tctctccaggcgggcgcc

SEQ ID NO: 19 - DNA, 25 bp, Artificial
  Primer 13
ggcgtcgtagataacgaggttcccg

SEQ ID NO: 20 - DNA, 21 bp, Artificial
  Primer 14
aacaacaacgacgtgtggggg

SEQ ID NO: 21 - DNA, 64 bp, Artificial
  Primer 15
ggggacaagtttgtacaaaaaagcaggctctaaacgtggggggggggacagtgtcctgctctcc

SEQ ID NO: 22 - DNA, 50 bp, Artificial
  Primer 16
ggggaccactttgtacaagaaagctgggtttaattaagactgcggcaccc

SEQ ID NO: 23 - DNA, 65 bp, Artificial
  Primer 17
ggggacaagtttgtacaaaaaagcaggctctaaacgtggggggggggacaatgtcctgctctccg

SEQ ID NO: 24 - DNA, 50 bp, Artificial
  Primer 18
ggggaccactttgtacaagaaagctgggtttatccaccattaacacggcg

SEQ ID NO: 25 - DNA, 2521 bp, Homo sapiens
catggggcccagggcaaagaccatctcctccctgttcttcctcctatgggtcctggctgagccggctgagaactcggacttctacctgcctggggattacctcctgggtggcctcttctccctccatgccaacatgaagggcattgttcaccttaacttcctgcaggtgcccatgtgcaaggagtatgaagtgaaggtgataggctacaacctcatgcaggccatgcgctttgcggtggaggagatcaacaatgacagcagcctgctgcctggtgtgctgctgggctatgagatcgtggatgtgtgctacatctccaacaatgtccagccggtgctctacttcctggcacacgaggacaacctccttcccatccaagaggactacagtaactacatttcccgtgtggtggctgtcattggccctgacaactccgagtctgtcatgactgtggccaacttcctctccctatttctccttccacagatcacctacagcgccatcagcgatgagctgcgagacaaggtgcgcttcccggctttgctgcgtaccacacccagcgccgaccaccacatcgaggccatggtgcagctgatgctgcacttccgctggaactggatcattgtgctggtgagcagcgacacctatggccgcgacaatggccagctgcttggcgagcgcgtggcccggcgcgacatctgcatcgccttccaggagacgctgcccacactgcagcccaaccagaacatgacgtcagaggagcgccagcgcctggtgaccattgtggacaagctgcagcagagcacagcgcgcgtcgtggtcgtgttctcgcccgacctgaccctgtaccacttcttcaatgaggtgctgcgccagaacttcactggcgccgtgtggatcgcctccgagtcctgggccatcgacccggtcctgcacaacctcacggagctgcgccacttgggcaccttcctgggcatcaccatccagagcgtgcccatcccgggcttcagtgagttccgcgagtggggcccacaggctgggccgccacccctcagcaggaccagccagagctatacctgcaaccaggagtgcgacaactgcctgaacgccaccttgtccttcaacaccattctcaggctctctggggagcgtgtcgtctacagcgtgtactctgcggtctatgctgtggcccatgccctgcacagcctcctcggctgtgacaaaagcacctgcaccaagagggtggtctacccctggcagctgcttgaggagatctggaaggtcaacttcactctcctggaccaccaaatcttcttcgacccgcaaggggacgtggctctgcacttggagattgtccagtggcaatgggaccggagccagaatcccttccagagcgtcgcctcctactaccccctgcagcgacagctgaagaacatccaagacatctcctggcacaccatcaacaacacgatccctatgtccatgtgttccaagaggtgccagtcagggcaaaagaagaagcctgtgggcatccacgtctgctgcttcgagtgcatcgactgccttcccggcaccttcctcaaccacactgaagatgaatatgaatgccaggcctgcccgaataacgagtggtcctaccagagtgagacctcctgcttcaagcggcagctggtcttcctggaatggcatgaggcacccaccatcgctgtggccctgctggccgccctgggcttcctcagcaccctggccatcctggtgatattctggaggcacttccagacacccatagttcgctcggctgggggccccatgtgcttcctgatgctgacactgctgctggtggcatacatggtggtcccggtgtacgtggggccgcccaaggtctccacctgcctctgccgccaggccctctttcccctctgcttcacaatctgcatctcctgtatcgccgtgcgttctttccagatcgtctgcgccttcaagatggccagccgcttcccacgcgcctacagctactgggtccgctaccaggggccctacgtctctatggcatttatcacggtactcaaaatggtcattgtggtaattggcatgctggccacgggcctcagtcccaccacccgtactgaccccgatgaccccaagatcacaattgtctcctgtaaccccaactaccgcaacagcctgctgttcaacaccagcctggacctgctgctctcagtggtgggtttcagcttcgcctacatgggcaaagagctgcccaccaactacaacgaggccaagttcatcaccctcagcatgaccttctatttcacctcatccgtctccctctgcaccttcatgtctgcctacagcggggtgctggtcaccatcgtggacctcttggtcactgtgctcaacctcctggccatcagcctgggctacttcggccccaagtgctacatgatcctcttctacccggagcgcaacacgcccgcctacttcaacagcatgatccagggctacaccatgaggagggactag

SEQ ID NO: 26 - DNA, 2559 bp, Homo sapiens
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccggtgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 27 - protein, 374 aa, Artificial
  G15Gi3
MARSLTWGCCPWCLTEEEKTAARIDQEINKILLEQKKQERGELKLLLLGPGESGKSTFIKQMRIIHGAGYSEEDRRAFRLLVYQNIFVSMQAMIEAMDRLQIPFSRPDSKQHASLVMTQDPYKVSSFEKPYAVAMQYLWRDAGIRACYERRREFHLLDSAVYYLSHLERIAEDDYIPTAQDVLRSRMPTTGINEYCFSVQKTKLRIVDVGGQKSERKKWIHCFENVIALIYLASLSEYDQCLEENSQENRMKESLALFSTILELPWFKSTSVILFLNKTDILEDKIHTSHLASYFPSFQGPRRDAEAAKRFILDMYARVYASCAEPHDGGRKGSRARRLFAHFTCATDTHSVRSVFKDVRDSVLARYLDECGLY

SEQ ID NO: 28 - protein, 113 aa, Artificial
  NAS variant
DSVLLSGQTLYAGASLTSGSYTLTIQNNCNLVKYQAGRQIWASDTDGQGSQCRLTLRSDGNLIIYDDNNMVVWGSDCWGNNGTYALVLQQDGLFVIYGPVLWPLGLNGCRSLN

SEQ ID NO: 29 - protein, 115 aa, Artificial
  NBS variant
DNVLLSGQTLAADASLQAGAYTLTIQNKCNLVKYQNGRQIWASNTDRRGSGCRLTLLSDGNLVIYDANNNDVWGSACWGDNGKYALVLQKDGRFVIYGPVLWSLGPNGCRRVNGG

SEQ ID NO: 30 - DNA, 33 bp, Artificial
  Primer 19
aaaaggcgcgccgccatggcccggtccctgact

SEQ ID NO: 31 - DNA, 50 bp, Artificial
  Primer 20
aaaagcggccgctcagtaaagcccacattcgtccaggtaccgggccagca

(19) United States
(12) Patent Application Publication
(10) Pub. No.: US 2013/0040371 A1
(43) Pub. Date: Feb. 14, 2013

(54) PROTEIN HAVING SWEETNESS

(71) Applicants: Keiko ABE, Warabi-shi (JP); Kenichiro Nakajima, Tokyo (JP); Tomiko Asakura, Tokyo (JP); Takumi Misaka, Nagareyama-shi (JP); Sho Ishii, Tokyo (JP)

(72) Inventors: Keiko ABE, Warabi-shi (JP); Kenichiro Nakajima, Tokyo (JP); Tomiko Asakura, Tokyo (JP); Takumi Misaka, Nagareyama-shi (JP); Sho Ishii, Tokyo (JP)

(21) Appl. No.: 13/544,675

(22) Filed: Jul. 9, 2012

Related U.S. Application Data
(62) Division of application No. 12/532,716, filed on Sep. 23, 2009, now abandoned.

(30) Foreign Application Priority Data
Mar. 23, 2007 (JP) 2007-076158

(51) Int. Cl. C12N 15/29; C12N 1/15; C12N 15/63

(52) U.S. Cl. 435/254.3; 536/23.6; 435/320.1

(57) ABSTRACT

Provided are: an NAS variant characterized by having sweetness independent of pH in the case of combination with a polypeptide NBS variant by causing substitution mutation or deletion mutation in one or several amino acids out of the amino acid residues constituting a polypeptide NAS; an NBS variant characterized by having sweetness independent of pH in the case of combination with a polypeptide NAS variant by causing substitution mutation or deletion mutation in one or several amino acids out of the amino acid residues constituting a polypeptide NBS; a dimer comprising a combination of the NAS variant and the NBS variant; a recombinant vector containing a gene encoding the NAS variant and a gene encoding the NBS variant; and a transformant containing the vector.

This application is a Divisional application of application Ser. No. 12/532,716, filed Sep. 23, 2009, the contents of which are incorporated herein by reference in their entirety. Ser. No. 12/532,716 is a National Stage Application, filed under 35 USC 371, of International (PCT) Application No. PCT/JP2008/051167, filed Jan. 28, 2008.

### TECHNICAL FIELD

The present invention relates to a protein having sweetness and applications thereof. More particularly, the present invention relates to a neoculin acidic subunit (NAS) variant and a neoculin basic subunit (NBS) variant each having sweetness independent of pH, a dimeric protein containing the variants, and a sweetener composition containing the dimeric protein.

### BACKGROUND ART

A protein having sweetness is considered to be useful because the protein can be a sweetener with lower calorie compared with sugars.

Curculigo latifolia is a plant which spontaneously grows in West Malaysia and southern part of Thailand and is classified into family Liliaceae. Neoculin contained in the plant is considered to be useful as a protein having a taste-modifying activity, e.g., an activity to give the sweetness to foods and drinks and an activity to give the sweetness to sour foods and drinks.

Neoculin is a heterodimeric protein and is formed of a polypeptide NAS and a polypeptide NBS.

The taste-modifying activity of neoculin has been already reported (e.g., see Patent Document 1). The case where neoculin exhibited intense sweetness was limited to the case where a sour substance was eaten or drunk after neoculin was taken in a mouth, i.e., the case under an acidic condition. Neoculin itself has sweetness even under a neutral condition, but intensity of its sweetness is weak. Thus, neoculin was excellent as a taste-modifying substance having, for example, an activity to give sweetness to sour foods, but was not suitable as a sweetener to be used for foods other than the sour foods.

The taste-modifying activity of neoculin remains in an oral cavity for a long period of time. From the reason as well, neoculin was not suitable for adding as a sweetener to foods.

In view of the above, development of a protein having sweetness independent of pH has been required.

Patent Document 1: WO 2005/073372 A

### DISCLOSURE OF THE INVENTION

### Problem to be Solved by the Invention

It is an object of the present invention to find a substance having sweetness independent of pH as above and provide a novel composition having sweetness containing the substance.

### Means for Solving the Problems

As a result of extensively studying based on the above problems, the inventors of the present invention have successfully found an NAS variant and an NBS variant which form a protein having a completely different nature from that of known neoculin by introducing mutations into several sites of amino acid residues in known polypeptides NAS and NBS.

Further, the inventors of the present invention have found that a dimeric protein containing the NAS variant and NBS variant is excellent in practicability as a sweetener, which is different from known neoculin, because the protein has sweetness independent of pH.

The inventors of the present invention have completed the present invention based on those findings.

That is, a first embodiment of the present invention relates to an NAS variant, comprising a polypeptide having substitution mutation or deletion mutation in one or several amino acids in amino acid residues constituting a polypeptide NAS, wherein the NAS variant has sweetness independent of pH when the NAS variant is combined with a polypeptide comprising an amino acid sequence shown in SEQ ID NO: 29.

A second embodiment of the present invention relates to an NAS variant according to the first embodiment, wherein the amino acids to be substituted or deleted are aspartic acid and/or histidine.

A third embodiment of the present invention relates to an NAS variant according to the first embodiment, wherein the amino acids to be substituted or deleted are histidine.

A fourth embodiment of the present invention relates to an NAS variant according to any one of the first to third embodiments, wherein substitution is substitution with at least one kind of amino acid selected from the group consisting of lysine, arginine, alanine, glycine, and serine.

A fifth embodiment of the present invention relates to an NAS variant according to any one of the first to third embodiments, wherein the substitution is substitution with alanine.

A sixth embodiment of the present invention relates to an NAS variant according to the first to fifth embodiments, which is glycosylated with an N-linked sugar chain comprising mannose/N-acetylglucosamine at a ratio of 9/2.

A seventh embodiment of the present invention relates to an NBS variant, comprising a polypeptide having substitution mutation or deletion mutation in one or several amino acids in amino acid residues constituting a polypeptide NBS, wherein the NBS variant has sweetness independent of pH when the NBS variant is combined with a polypeptide comprising an amino acid sequence shown in SEQ ID NO: 28.

An eighth embodiment of the present invention relates to an NBS variant according to the seventh embodiment, wherein the amino acids to be substituted or deleted are aspartic acid and/or histidine.

A ninth embodiment of the present invention relates to an NBS variant according to the seventh embodiment, wherein the amino acids to be substituted or deleted are histidine.

A tenth embodiment of the present invention relates to an NBS variant according to any one of the seventh to ninth embodiments, wherein substitution is substitution with at least one kind of amino acid selected from the group consisting of lysine, arginine, alanine, glycine, and serine.

An eleventh embodiment of the present invention relates to an NBS variant according to any one of the seventh to ninth embodiments, wherein the substitution is substitution with alanine.

A twelfth embodiment of the present invention relates to a dimeric protein, comprising an arbitrary combination of any one of the polypeptides out of the NAS variants according to the first to sixth embodiments and any one of the polypeptides out of the NBS variants according to the seventh to eleventh embodiments, wherein the dimeric protein has sweetness independent of pH.

A thirteenth embodiment of the present invention relates to a dimeric protein, comprising a combination of an NAS variant in which one or several histidines constituting a polypeptide NAS are deleted or substituted with alanines and an NBS variant in which one or several histidines constituting a polypeptide NBS are deleted or substituted with alanines, wherein the dimeric protein has sweetness independent of pH.

A fourteenth embodiment of the present invention relates to a sweetener composition, comprising one kind or two or more kinds selected from the group consisting of the NAS variants according to the first to sixth embodiments, the NBS variants according to the seventh to eleventh embodiments, and the dimeric proteins according to the twelfth to thirteenth embodiments as active components, wherein the sweetener composition has sweetness independent of pH.

A fifteenth embodiment of the present invention relates to genes encoding the NAS variants according to the first to fifth embodiments.

A sixteenth embodiment of the present invention relates to recombinant vectors comprising the genes according to the fifteenth embodiment.

A seventeenth embodiment of the present invention relates to genes encoding the NBS variants according to the seventh to eleventh embodiments.

An eighteenth embodiment of the present invention relates to recombinant vectors comprising the genes according to the seventeenth embodiment.

A nineteenth embodiment of the present invention relates to a transformant carrying the recombinant vectors according to the sixteenth and/or eighteenth embodiments.

### Effect of the Invention

According to the present invention, the novel polypeptide NAS variant and polypeptide NBS variant, which are different from the known polypeptide NAS and polypeptide NBS, and have the novel amino acid sequences are provided, and using the polypeptides, novel dimeric protein having sweetness independent of pH can be provided.

Further, by using the dimeric protein, a novel sweetener composition which is practically applied to foods and has sweetness independent of pH can be provided.

The dimeric protein provided by the present invention has a characteristic of “having sweetness independent of pH”, which is completely different from that of known neoculin.

Generally, protein has a deep association with its function and its structure. A mutation in an amino acid sequence, i.e., the primary structure, sometimes causes a drastic change in the structure of the protein, and as a result, the mutation may lead to a protein having a completely different nature.

Thus, the NAS variant, the NBS variant, and the dimeric protein formed thereof, which are provided in the present invention, are different from the known polypeptide NAS and polypeptide NBS, and neoculin only in one or several sites in the sequence in the primary structure, but it is speculated that the NAS variant, the NBS variant, and the dimeric protein are completely different from NAS, NBS, and neoculin in structure.

### BRIEF DESCRIPTION OF DRAWINGS

FIG. 1 is a view illustrating a speculated change in a neoculin structure;

FIG. 2 is a view illustrating an estimated sugar chain structure of a sugar chain of an NAS variant;

FIG. 3 is a view illustrating an outline of a method of preparing an expression vector for the NAS variant;

FIG. 4 is a view illustrating an outline of a method of preparing an expression vector for an NBS variant;

FIG. 5 is a view showing an elution pattern when a dimeric protein of the present invention was purified by phenyl hydrophobic column chromatography;

FIG. 6 is a view showing an elution pattern when the dimeric protein of the present invention was purified by gel filtration column chromatography;

FIG. 7 is a graph illustrating results of sensory evaluation of sweetness intensity;

FIG. 8 is a graph illustrating results of evaluation of a remaining taste-modifying activity of neoculin;

FIG. 9 is a graph illustrating results of evaluation of a remaining taste-modifying activity of the dimeric protein of the present invention;

FIG. 10 is a graph illustrating results of evaluation of sweetness intensity by response of cells to the dimeric protein of the present invention; and

FIG. 11 is a graph illustrating results of evaluation of sweetness intensity by response of cells to neoculin.

### BEST MODE FOR CARRYING OUT THE INVENTION

### (1) NAS Variant of the Present Invention

The NAS variant of the present invention refers to an NAS variant, comprising a polypeptide having substitution mutation or deletion mutation in one or several amino acids in amino acid residues constituting a known polypeptide NAS, wherein the NAS variant has sweetness independent of pH when the NAS variant is combined with a polypeptide comprising an amino acid sequence shown in SEQ ID NO: 29.

In the present invention, the polypeptide NAS is one of two subunits constituting neoculin and is shown below in the (A) or (B):
(A) a polypeptide comprising the amino acid sequence shown in SEQ ID NO: 1 in the sequence listing;
(B) a polypeptide which comprises an amino acid sequence with the substitution, deletion, insertion, addition, or inversion of one or several amino acids in the amino acid sequence shown in SEQ ID NO: 1 in the sequence listing, and can form, together with a polypeptide NBS, neoculin as a dimeric protein having a taste-modifying activity.

Here, the phrase “taste-modifying activity” means an activity of prominently reducing sourness, bitterness, or astringency as well as enhancing the taste of foods or drinks. Specifically, the activity means an activity suppressing the bitterness of bitter foods or drinks, an activity suppressing the astringency of foods or drinks with astringent taste, an activity giving sweetness to foods or drinks, an activity giving sweetness to sour foods or drinks, and an activity suppressing the sourness of sour foods or drinks.

In addition, in the present invention, the polypeptide NBS is one of two subunits constituting neoculin and is shown below in the (C) or (D):
(C) a polypeptide comprising the amino acid sequence shown in SEQ ID NO: 2 in the sequence listing;
(D) a polypeptide which comprises an amino acid sequence with the substitution, deletion, insertion, addition, or inversion of one or several amino acids in the amino acid sequence shown in SEQ ID NO: 2 in the sequence listing, and can form, together with a polypeptide NAS, neoculin as a dimeric protein having a taste-modifying activity.

It should be noted that details of the aforementioned polypeptides NAS and NBS are described in WO 2005/073372, and the polypeptides and genes thereof can be acquired easily by the methods described therein.

The NAS variant of the present invention is a polypeptide having substitution mutation or deletion mutation in one or several amino acids in the amino acid residues constituting the above known polypeptide NAS. That is, the NAS variant of the present invention is a polypeptide obtained by introducing the substitution mutation or deletion mutation in one or several amino acids in the amino acid sequence of the known polypeptide NAS.

The inventors of the present invention carried out X-ray crystallographic analysis and molecular dynamic simulation of neoculin, and reported their results in Journal of Molecular Biology, Vol. 359, pages 148-158, 2006. According to the report, it has been speculated that as shown in FIG. 1, neoculin has a state in which two subunits of the dimeric protein are close to each other under neutral conditions whereas the structure of neoculin changes to a state in which two subunits are mutually repulsive under acidic conditions. That is, it has been believed that neoculin exhibits intense sweetness in a state where the two subunits are mutually repulsive. It has been also suggested that an amino acid having a side chain which changes a charge in the region from the neutral to the acidity is involved in the structural change.

From the above finding, the type of the amino acid in which the substitution mutation or the deletion mutation is introduced in the polypeptide NAS is not limited in the present invention as long as the amino acid receives a structural change to the state where the two subunits constituting the dimeric protein are mutually repulsive even in the neutral region. However, the amino acids having the side chain which changes the charge in the region from the neutral to the acidity, i.e., aspartic acid and histidine, are desirable and in particular, histidine is preferred.

The amino acids having the mutation are one or several amino acids, preferably 1 to 10 amino acids, and more preferably 2 amino acids in the amino acid residues constituting the polypeptide NAS, in the NAS variant of the present invention.

When the above mutation is substitution, the type of the amino acid which is substituted for the amino acid such as aspartic acid or histidine constituting the polypeptide NAS and is contained newly is not limited as long as the aforementioned structural change is induced. Particularly preferred are lysine and arginine which have a positive charge even under neutral conditions, and alanine, glycine, and serine which have lower molecular weights and smaller side chains and thus hardly cause steric hindrance.

A combination of the amino acid to be substituted and the amino acid to be newly contained is also not limited, and for example, the substitution of histidine at position 14 with alanine and the substitution of histidine at position 36 with alanine in the NAS polypeptide chain are included. The amino acid sequence of the NAS variant obtained from the result is shown in SEQ ID NO: 28 in the sequence listing.

The NAS variant of the present invention is preferably glycosylated with a sugar chain, particularly preferably an N-linked sugar chain because the NAS variant can form a dimeric protein with a higher stability. Herein, the N-linked sugar chain means the general name of a sugar chain structure extending from N-acetylglucosamine, as the start point, bound to the asparagine residue existing in the primary structure of protein.

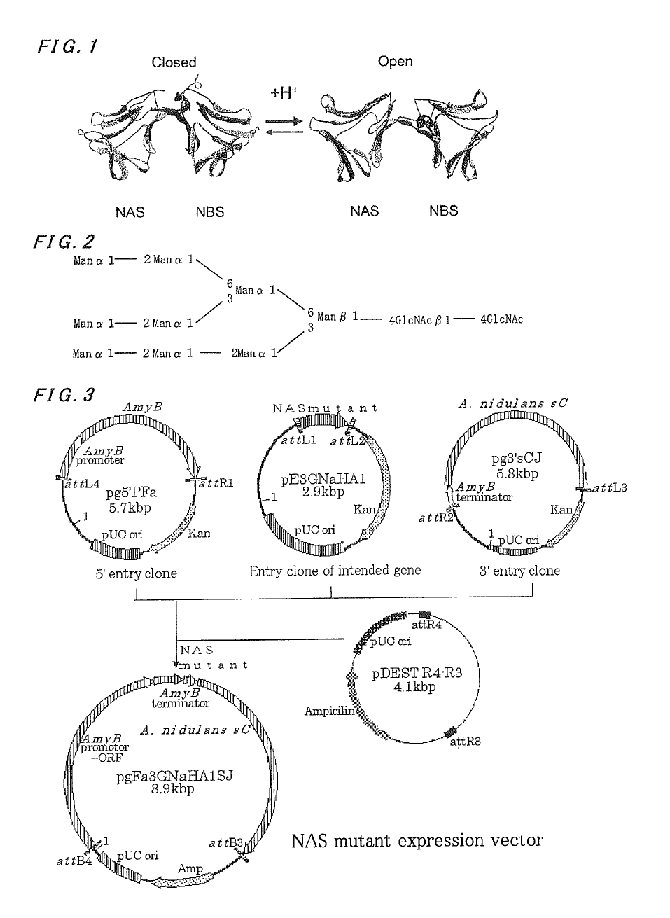

Among the N-linked sugar chains, an N-linked sugar chain comprising mannose/N-acetylglucosamine at a ratio of 9/2 is preferable. Specifically, the N-linked sugar chain is preferably a sugar chain in the structure shown in FIG. 2. Furthermore, apart of the structure shown in FIG. 2 may have addition, deletion, substitution or modification.

From the standpoint of the binding feature of such N-linked sugar chain, the binding site of the N-linked sugar chain in the NAS variant may possibly be asparagine at position 81 in the amino acid sequence shown in SEQ ID NO: 1 in the sequence listing.

The NAS variant of the present invention is the polypeptide obtained by introducing the mutation as above into the known polypeptide NAS, and can be obtained by combining publicly known gene recombination technology, technique for screening of variants, and technique for protein expression.

For example, a DNA sequence of a gene encoding the intended NAS variant can be obtained by introducing mutation into the DNA sequence of the NAS gene shown in SEQ ID NO: 1 in the sequence listing using a publicly known inverse PCR method as described in Example 1. The NAS variant of the present invention can be obtained by subjecting the DNA sequence to a publicly known appropriate protein expression system.

The NAS variant of the present invention is characterized by having sweetness independent of pH when combined with the polypeptide comprising the amino acid sequence shown in SEQ ID NO: 29. The polypeptide comprising the amino acid sequence shown in SEQ ID NO: 29 is one of the NBS variants of the present invention described later. And the dimeric protein of the present invention described later is formed by combining the NAS variant of the present invention with the polypeptide comprising the amino acid sequence shown in SEQ ID NO: 29.

“Having sweetness independent of pH” in the present invention means having intense sweetness in not only the acidic region but also the neutral region and the basic region. That is, the present invention is different from those such as known neoculin having sweetness intensity which is remarkably reduced in the neutral region compared with the acidic region.

Specifically, the sweetness intensity corresponding to the intensity in the case of “having sweetness independent of pH” in the present invention is necessary to be larger than 12.5, is preferably 25 or more, and more preferably 50 or more in any region of the acidic, neutral, and basic regions.

The sweetness intensity is represented by a relative value when the sweetness intensity of aspartame is 1, and the sweetness intensity per molar concentration is here compared by sensory evaluation.

The NAS variant of the present invention may be a variant which, together with an NBS variant other than the polypeptide comprising the amino acid sequence shown in SEQ ID NO: 29, also forms the dimeric protein and exerts sweetness independent of pH as the dimeric protein.

A possibility is not excluded that the NAS variant of the present invention forms the dimeric protein having sweetness independent of pH as a heterodimer with the known polypeptide NBS or polypeptide NAS, or a homodimer of the NAS variants.

Further the NAS variant of the present invention may exert sweetness independent of pH as a monomer.

### (2) NBS Variant of the Present Invention

The NBS variant of the present invention refers to an NBS variant, comprising a polypeptide having substitution mutation or deletion mutation in one or several amino acids in amino acid residues constituting a known polypeptide NBS, wherein the NBS variant has sweetness independent of pH when the NBS variant is combined with a polypeptide comprising an amino acid sequence shown in SEQ ID NO: 28.

Further, the polypeptide NBS mentioned here is as described in the above (1).

The NBS variant of the present invention is a polypeptide having substitution mutation or deletion mutation in one or several amino acids in the amino acid residues constituting the above known polypeptide NBS. That is, the NBS variant of the present invention is a polypeptide obtained by introducing the substitution mutation or deletion mutation in one or several amino acids in the amino acid sequence of the known polypeptide NBS.

Like the NAS variant of the present invention, the type of the amino acid in which the substitution mutation or the deletion mutation is introduced in the polypeptide NBS is not limited in the present invention as long as the amino acid receives a structural change to the state where the two subunits constituting the dimeric protein are mutually repulsive even in the neutral region. However, the amino acids having the side chain which changes the charge in the region from the neutral to the acidity, i.e., aspartic acid and histidine, are desirable and in particular, histidine is preferred.

The amino acids having the mutation are one or several amino acids, preferably 1 to 11 amino acids, and more preferably 3 amino acids in the amino acid residues constituting the polypeptide NBS, in the NBS variant of the present invention.

When the above mutation is substitution, the type of the amino acid which is substituted for the amino acid such as aspartic acid or histidine constituting the polypeptide NBS and is contained newly is not limited as long as the aforementioned structural change is induced. Particularly preferred are lysine and arginine which have a positive charge even under neutral conditions, and alanine, glycine, and serine which have lower molecular weights and smaller side chains and thus hardly cause steric hindrance.

A combination of the amino acid to be substituted and the amino acid to be newly contained is also not limited, and for example, the substitution of histidine at position 11 with alanine, the substitution of histidine at position 14 with alanine, and the substitution of histidine at position 67 with alanine in the NBS polypeptide chain are included. The amino acid sequence of the NBS variant obtained from the result is shown in SEQ ID NO: 29 in the sequence listing.

The NBS variant of the present invention is the polypeptide obtained by introducing the mutation as above into the known polypeptide NBS, and can be obtained by combining publicly known gene recombination technology, technique for screening of variants, and technique for protein expression.

For example, a DNA sequence of a gene encoding the intended NBS variant can be obtained by introducing mutation into the DNA sequence of the NBS gene shown in SEQ ID NO: 2 in the sequence listing using a publicly known inverse PCR method as described in Example 2. The NBS variant of the present invention can be obtained by subjecting the DNA sequence to a publicly known appropriate protein expression system.

The NBS variant of the present invention is characterized by having sweetness independent of pH when combined with the polypeptide comprising the amino acid sequence shown in SEQ ID NO: 28. The polypeptide comprising the amino acid sequence shown in SEQ ID NO: 28 is one of the NAS variants of the present invention described above. And the dimeric protein of the present invention described later is formed by combining the NBS variant of the present invention with the polypeptide comprising the amino acid sequence shown in SEQ ID NO: 28.

Here, the phrase “having sweetness independent of pH” has the same meaning as that described in the above (1).

The NBS variant of the present invention may be a variant which, together with an NAS variant other than the polypeptide comprising the amino acid sequence shown in SEQ ID NO: 28, also forms the dimeric protein and exerts sweetness independent of pH as the dimeric protein.

A possibility is not excluded that the NBS variant of the present invention forms the dimeric protein having sweetness independent of pH as a heterodimer with the known polypeptide NAS or polypeptide NBS, or a homodimer of the NBS variants.

Further, the NBS variant of the present invention may exert sweetness independent of pH as a monomer.

### (3) Dimeric Protein of the Present Invention

The dimeric protein of the present invention is a dimeric protein, characterized by comprising an arbitrary combination of any one of the polypeptides out of the NAS variants described in (1) and any one of the polypeptides out of the NBS variants described in (2), and having sweetness independent of pH.

Here, the phrase “having sweetness independent of pH” has the same meaning as that described in the above (1).

The dimeric protein of the present invention can be obtained by using a gene encoding the NAS variant and/or a gene encoding the NBS variant and subjecting the genes to a publicly known appropriate protein expression system. For example, the dimeric protein of the present invention can be obtained according to the method described in the following (6) by subjecting the genes to the protein expression system using Aspergillus oryzae reported in Example 11 in WO 2005/073372 previously filed by the applicants of the present invention.

### (4) Sweetener Composition of the Present Invention

The sweetener composition of the present invention is characterized by comprising one kind or two or more kinds selected from the group consisting of the NAS variants described in (1), the NBS variants described in (2), and the dimeric proteins described in (3) as active components, and having sweetness independent of pH.

The NAS variant in the above (1) and the NBS variant in the above (2) form a heterodimer to become the dimeric protein of the present invention having sweetness independent of pH as described above. When the NAS variant or the NBS variant exerts sweetness independent of pH as the homodimer or the monomer thereof, the sweetener composition of the present invention also includes those containing the NAS variant alone or the NBS variant alone.

The sweetener composition of the present invention may be incorporated as it is but may be added at an appropriate amount to foods or drinks including vegetable juice, fruit juices of for example grapefruit, and various seasoning liquids for cooking for sushi neta (sushi topping), or pharmaceutical agents or the like. The blended amount of neoculin variant in this case is for example 5 to 5,000 μg/ml, particularly preferably 50 to 500 μg/ml, when the composition containing a neoculin variant powder highly purified is added to a drink.

Additionally, the sweetener composition of the present invention may be used after processing into the form of powder, solution, sheet, spray, granule or emulsion, depending on the property of a food or a drink or a pharmaceutical agent as a blend subject.

### (5) Genes of the Present Invention

The genes of the present invention are the gene encoding the

NAS variant described in (1) and the gene encoding the NBS variant described in (2).

That is, firstly, the gene encoding the NAS variant of the present invention is a gene encoding the NAS variant, which is characterized by comprising a polypeptide having substitution mutation or deletion mutation in one or several amino acids in amino acid residues constituting a polypeptide NAS, and having sweetness independent of pH when the NAS variant is combined with a polypeptide comprising the amino acid sequence shown in SEQ ID NO: 29.

That is, the gene encoding the NAS variant of the present invention can be obtained by introducing the mutation into the gene encoding the known polypeptide NAS so that the substitution mutation or the deletion mutation occurs in one or several amino acid residues.

The mutation introduced into the amino acid sequence of the polypeptide NAS is as described in the above (1).

The gene encoding the polypeptide NAS may be obtained as (a) DNA containing a nucleotide sequence comprising the nucleotides 70 to 408 in the nucleotide sequence shown in SEQ ID NO: 5 in the sequence listing, and may be obtained as DNA hybridizing with the DNA of a nucleotide sequence substantially identical to the aforementioned nucleotide sequence, namely (b) the DNA of the nucleotide sequence comprising the nucleotides 70 to 408 in the nucleotide sequence shown in SEQ ID NO: 5 in the sequence listing or a nucleotide sequence capable of functioning as probe prepared from at least a part of the aforementioned nucleotide sequence, under stringent conditions, and encoding a polypeptide capable of forming neoculin as a dimeric protein having a taste-modifying activity together with the polypeptide NBS.

Here, the phrase “stringent conditions” means conditions under which so-called specific hybrid is formed but non-specific hybrid is not formed. It is difficult to numerically express the conditions clearly. Nonetheless, one example thereof is a condition under which DNAs with high homology, for example, 90% or more homology, are hybridized together, while DNAs with lower homology are never hybridized, and a rinse condition for general hybridization, for example, a rinse condition of 0.1×SSC at a salt concentration corresponding to 0.1% SDS and at 65° C.

The gene encoding the polypeptide NAS can be obtained by the following method:

For example, mRNA is extracted from a fruit of Curculigo latifolia several weeks after pollination, cDNA is synthetically prepared with reverse transcription polymerase chain reaction (RT-PCR), and the cDNA is packaged in a phage vector. Then, infection with the phage vector is carried out to obtain a cDNA library. Subsequently, a probe prepared on the basis of the amino acid sequence of the polypeptide NAS (see SEQ ID NO: 1 in the sequence listing, for example) is allowed to identify the intended DNA with a plaque hybridization, and the intended DNA is recovered and obtained.

The DNA may also be obtained by PCR using, as a primer, an oligonucleotide synthetically prepared on the basis of the nucleotide sequence comprising the nucleotides 70 to 408 in the nucleotide sequence described as SEQ ID NO: 5 in the sequence listing. Otherwise, the DNA shown above in (a) or (b) may be synthetically prepared with various commercially available DNA synthesizers.

Additionally, the DNA shown in (b) may be obtained, for example, by site-directed mutagenesis including appropriately introducing mutations such as substitution, deletion, insertion or addition into the nucleotide sequence comprising the nucleotides 70 to 408 in the nucleotide sequence shown in SEQ ID NO: 5 in the sequence listing. The DNA may also be obtained by known mutation processes.

It should be noted that the method of introducing mutation into the gene encoding the polypeptide NAS so that the substitution mutation or the deletion mutation occurs in one or several amino acid residues is also as described in the above.

In addition, secondly, the gene encoding the NBS variant of the present invention is a gene encoding the NBS variant, which is characterized by comprising a polypeptide having substitution mutation or deletion mutation in one or several amino acids in amino acid residues constituting a polypeptide NBS, and having sweetness independent of pH when the NBS variant is combined with a polypeptide comprising the amino acid sequence shown in SEQ ID NO: 28.

That is, the gene encoding the NBS variant of the present invention can be obtained by introducing the mutation into the gene encoding the known polypeptide NBS so that the substitution mutation or the deletion mutation occurs in one or several amino acid residues.

The mutation introduced into the amino acid sequence of the polypeptide NBS is as described in the above (1).

The gene encoding the polypeptide NBS may be obtained as (c) DNA containing a nucleotide sequence comprising the nucleotides 77 to 421 in the nucleotide sequence shown in SEQ ID NO: 12 in the sequence listing, and may be obtained as DNA hybridizing with the DNA of a nucleotide sequence substantially identical to the aforementioned nucleotide sequence, namely (d) the DNA of the nucleotide sequence comprising the nucleotides 77 to 421 in the nucleotide sequence shown in SEQ ID NO: 12 in the sequence listing or a nucleotide sequence capable of functioning as probe prepared from at least a part of the aforementioned nucleotide sequence, under stringent conditions, and encoding a polypeptide capable of forming neoculin as a dimeric protein having a taste-modifying activity together with the polypeptide NAS.

The phrase “stringent conditions” has the same meaning as that described for the gene encoding the polypeptide NAS.

The gene encoding the polypeptide NBS can be obtained, for example, in the same manner as in the case of the polypeptide NAS from a fruit of Curculigo latifolia several weeks after pollination. Additionally, the DNA shown above in (c) or (d) may also be obtained by PCR using, as a primer, an oligonucleotide synthetically prepared on the basis of the nucleotide sequence of the nucleotides 77 to 421 in the nucleotide sequence shown in SEQ ID NO: 12, or may be synthesized with various commercially available DNA synthesizers.

Additionally, the DNA shown in (d) may also be obtained by site-directed mutagenesis including appropriately introducing mutations such as substitution, deletion, insertion or addition into the nucleotide sequence comprising the nucleotides 77 to 421 in the nucleotide sequence shown in SEQ ID NO: 12 in the sequence listing. Additionally, the DNA may be obtained by known mutation processes.

The method of introducing the mutation into the gene encoding the polypeptide NBS so that the substitution mutation or the deletion mutation occurs in one or several amino acids is also as described in the above.

It is needless to say that the two genes of the present invention may satisfactorily contain a regulatory element for the nucleotide sequences and structural genes.

### (6) Recombinant Vectors and Transformants of the Present Invention

In accordance with the invention, a recombinant vector carrying the gene encoding the NAS variant as well as a recombinant vector carrying the gene encoding the NBS variant are provided for a host-vector system for highly efficiently expressing the genes encoding the NAS variant and the NBS variant to industrially produce the dimeric protein of the present invention. In accordance with the invention, further, a transformant harboring any one type of or a combination of the two types of recombinant vectors is also provided.

The dimeric protein of the present invention can be produced by culturing the above transformant harboring two types of recombinant vectors. Further, the dimeric protein of the present invention can also be produced by binding the NAS variant obtained by culturing the transformant harboring the recombinant vector in which the NAS variant gene has been introduced and the NBS variant obtained by culturing the transformant harboring the recombinant vector in which the NBS variant gene has been introduced.

The recombinant vector of the present invention is required to have an expression and regulation functions including a promoter function so as to express the gene encoding the NAS variant or the gene encoding the NBS variant.

Additionally, the recombinant vectors preferably have such a function that the NAS variant and the NBS variant generated via the expression of the NAS variant gene and the NBS variant gene introduced into the vectors inside the host are secreted from the host cells. In addition to the genes encoding the NAS variant and the NBS variant, specifically, a gene encoding a protein secreted by a host, for example a gene of a secretory protein a-amylase in a koji mold in case of Aspergillus oryzae as a host, is integrated in an appropriate vector to express the genes for expression in a form of a fusion protein of the NAS variant with α-amylase and the NBS variant with α-amylase, whereby the NAS variant and the NBS variant can be secreted and generated extracellularly from the host cells. Instead of the α-amylase, glucoamylase (GlaA) may also be used.

In case of the expression in the form of the fusion protein as described above, preferably, the recombinant vectors of the present invention have a processing function working for altering the fusion protein into the NAS variant or the NBS variant alone. Specifically, for example, utilizing a nucleotide sequence encoding an amino acid sequence (Lys-Arg, Lys-Lys, Arg-Lys, Arg-Arg) recognized by a KEX2-like protease existing in the Golgi body of a koji mold, the nucleotide sequence is integrated and expressed in between the α-amylase gene and the NAS variant gene and between the α-amylase gene and the NBS variant gene, and the fusion gene is expressed. After the expression, KEX2-like protease digestion is carried out to isolate a-amylase from the resulting fusion protein generated by the transformant, to obtain the dimeric protein of the NAS variant and the NBS variant.

For cleavage with KEX2-like protease as described above, possibly, the cleavage may sometimes be inaccurate, depending on the structure around the recognition sequence by KEX2, and the structure around the cleavage site may be complicated as the dimeric protein of the present invention forms heterodimer. Therefore, the cleavage efficiency and the accuracy of the cleavage may preferably be improved. For example, about three residues of amino acids with lower molecular weights and smaller side chains such as glycine, alanine and serine to hardly cause steric hindrance can be inserted immediately before aspartic acid at the N termini of the NAS variant and the NBS variant.

Such a series of procedures may be done in a simpler manner utilizing a vector construction kit (Multisite Gateway Three-Fragment Vector Construction Kit; manufactured by Invitrogen). In other words, the construction is done in the order of (1) the preparation of 5′ entry clone, (2) the preparation of the entry clone of an intended gene, (3) the preparation of 3′ entry clone, (4) the preparation of a recombinant vector from a recombination of the three types of entry clones and destination vector, utilizing the kit, to prepare an intended recombinant vector. Based on general methods, alternatively, a necessary gene fragment is excised and then introduced into an appropriate site on a vector, to prepare a recombinant vector.

Specific examples of the recombinant vector in accordance with the invention include pgFa3GNaHA1SJ as a recombinant vector with the gene encoding the NAS variant introduced therein, and pgFa3GNbHA1Ta as a recombinant vector with the gene encoding the NBS variant introduced therein (see Example 3).

Subsequently, the transformant of the invention is now described. The transformant of the invention is prepared by integrating the gene encoding the NAS variant and/or the gene encoding the NBS variant into an appropriate host. In other words, the transformant of the present invention includes a recombinant vector containing the gene encoding the NAS variant and/or a recombinant vector containing the gene encoding the NBS variant.

The host for the recombinant vector in accordance with the invention is preferably eukaryote. Prokaryotes such as Escherichia coli are not preferable as the hosts. Because the protein is generated as an inclusion body in the bacterial cell, so as to alter the inclusion body into the accurate heterodimer capable of exerting sweetness, therefore, the NAS variant and NBS variant constituting the inclusion body are required to be once solubilized using a solubilization agent such as guanidine chloride and then reconstructed (reconstituted). When eukaryotes such as koji mold are used as the host, in contrast, the use of reagents such as solubilization agent is not required. With no need of extra processes such as reconstruction, additionally, the protein can be secreted and generated as a dimeric protein having sweetness independent of pH.

The host for introducing the recombinant vector therein in accordance with the invention is preferably a filamentous fungus among eukaryotes, more preferably koji mold among them. Specifically, koji mold belonging to Aspergillus oryzae, typically an Aspergillus oryzae strain NS-tApE is preferable.

As an example of the transformant, the inventors of the present invention obtained an Aspergillus oryzae strain NStApE-NCLHA by introducing a recombinant vector containing the NAS variant gene and a recombinant vector containing the NBS variant gene into the Aspergillus oryzae strain NS-tApE. The Aspergillus oryzae strain NStApE-NCLHA has been deposited on Mar. 13, 2007 under accession No. FERM BP-10799 at the International Patent Organism Depositary, the National Institute of Advanced Industrial Science and Technology, Chuo-6, 1-1, Higashi 1-chome, Tsukuba-shi, Ibaraki-ken, Japan.

The dimeric protein of the present invention, which is a dimeric protein having sweetness independent of pH can be produced by culturing the transformant.

The transformant is preferably cultured under conditions such that the expression ratio of the gene encoding the NAS variant and the gene encoding the NBS variant may be a specific ratio. When either one of the expression levels is one-sided, the efficiency of the generated heterodimer comprising the NAS variant and NBS variant is lower, because homodimers are also formed. Thus, the production efficiency of the dimeric protein of the present invention is then lowered.

In accordance with the invention, the ratio of the gene encoding the NAS variant and the gene encoding the NBS variant for use in the transformation procedure is set at 1:5. In that case, a strain with a high expression level could be screened in the resulting transformants with high efficiency. So as to achieve a high production efficiency, culturing is done preferably under a condition of a culture medium around pH 8.0.

In such a manner, the dimeric protein of the present invention can be produced efficiently.

The dimeric protein of the present invention can also be obtained by separately culturing the transformant integrating the gene encoding the NAS variant or the transformant integrating the gene encoding the NBS variant to separately produce the NAS variant or the NBS variant, and subsequently binding the obtained both subunits. At that time, the method of producing the transformants and the culture method thereof can be the same as the above.

### EXAMPLES

Subsequently, the best modes for carrying out the invention is described in detail with reference to examples, but the present invention is not limited to the following examples.

### Example 1

A plasmid (hereinafter referred to as pNAS) containing a gene encoding an NAS polypeptide was produced. Subsequently, a plasmid (hereinafter referred to as pNASH14AH36A) containing a gene encoding an NAS variant in which mutations had been introduced using an Inverse PCR method was obtained. The mutations were introduced into the gene so that histidine at position 14 was substituted with alanine and histidine at position 36 was substituted with alanine in a polypeptide chain.

mRNA was extracted from a fruit of Curculigo latifolia and a cDNA library was produced. Then, screening was performed using an NAS gene fragment as a probe, and a cDNA clone of the NAS gene was obtained. A nucleotide sequence of the obtained cDNA was as shown in SEQ ID NO: 5 of the sequence listing. Detail techniques are disclosed in Example 6 in WO 2005/073372.

Subsequently, using the gene region encoding a mature NAS polypeptide (a part comprising the nucleotides 70 to 408 in the nucleotide sequence shown in SEQ ID NO: 5 in the sequence listing) as template among the cDNA clones, PCR was done using primer 1 comprising the nucleotide sequence shown in SEQ ID NO: 6 in the sequence listing (5′-CGGGATCCGGACAGTGTCCTGCTCTCC-3′) and primer 2 comprising the nucleotide sequence shown in SEQ ID NO: 7 in the sequence listing (5′-CCGCTCGAGTTAATTAAGACTGCGGCACCC-3′). A resulting DNA fragment was introduced into pBluescript II (SK-) to yield an NAS gene clone (pNAS).

In addition, a DNA sequence encoding histidine at position 14 in an NAS polypeptide on pNAS was substituted with a sequence encoding alanine. Specifically, PCR with pNAS as the template was carried out using a primer 3 (5′-ggcGCCGGCATACAGAGTTTGCC-3′; small letters denote a mutated region) shown in SEQ ID NO: 8 in the sequence listing and a primer 4 (5′-TCCCTCACGTCGGGCAGCTATA-3′) designed to anneal in a reverse direction to an adjacent region and shown in SEQ ID NO: 9 in the sequence listing. The primers used at that time were previously phosphorylated with T4 polynucleotide kinase. A reaction composition solution and reaction conditions for PCR are as follows.

Reaction composition solution (per 20 μL)

| | | |
|---|---|---|
| 5× iProof buffer | 4 | μL |
| 10 mM dNTP | 1.6 | μL |
| Phosphorylated primer 3 (10 pmol/μL) | 1 | μL |
| Phosphorylated primer 4 (10 pmol/μL) | 1 | μL |
| pNAS (1 ng/μL) | 1 | μL |
| iProof high-fidelity DNA polymerase (BioRad) | 0.2 | μL |
| H₂O | 11.2 | μL |

PCR conditions
98° C. for 30 seconds×1 cycle
(98° C. for 30 seconds, 55° C. for 30 seconds and 72° C. for 55 seconds)×30 cycles
72° C. for 10 minutes×1 cycle
Then, cooled to 4° C.

A resulting fragment amplified by PCR was purified and self-ligation was performed by a ligation reaction to yield a vector containing the gene having the introduced mutation. The vector was introduced into Escherichia coli DH5α strain, and a plasmid was extracted from the resulting colony.

The same manipulation as the above was repeated in the resulting plasmid to substitute the DNA sequence encoding histidine at position 36 in the NAS polypeptide with the sequence encoding alanine. At that time, a primer 5 (5′-ggcCTGGTATTTCACCAGGTTGCAG-3′ ; the small letters denote the mutated region) shown in SEQ ID NO: 10 in the sequence listing and a primer 6 (5′-GGGAGGCAGATCTGGGCTA-3′) designed to anneal in the reverse direction to the adjacent region and shown in SEQ ID NO: 11 in the sequence listing were used. As described above, a plasmid (pNASH14AH36A) containing the gene encoding the NAS variant was obtained.

### Example 2

A plasmid (hereinafter referred to as pNBS) containing a gene encoding NBS was produced. Subsequently, a plasmid (hereinafter referred to as pNBSH11AH14AH67A) containing a gene encoding an NBS variant in which mutations had been introduced using an Inverse PCR method was obtained. The mutations were introduced into the gene so that histidine at position 11 was substituted with alanine, histidine at position 14 was substituted with alanine, and histidine at position 67 was substituted with alanine in a polypeptide chain.

mRNA was extracted from a fruit of Curculigo latifolia and a cDNA library was produced. Then, screening was performed using an NBS gene fragment as a probe, and a cDNA clone of the NBS gene was obtained. A nucleotide sequence of the obtained cDNA was as shown in SEQ ID NO: 12 in the sequence listing. Detail techniques are disclosed in Examples 1 to 11 in JP-A-6-189771.

Subsequently, using the gene region encoding the mature NBS (a part comprising the nucleotides 77 to 421 in the nucleotide sequence shown in SEQ ID NO: 12 in the sequence listing) as template among the cDNA clones, PCR was done using primer 7 comprising the nucleotide sequence shown in SEQ ID NO: 13 in the sequence listing (5′-CGGGATCCGGACAATGTCCTGCTCTCC-3′) and primer 8 comprising the nucleotide sequence shown in SEQ ID NO: 14 in the sequence listing (5′-CCGCTCGAGTTATCCACCATTAACACGGCG-3′). A resulting DNA fragment was introduced into pBluescript II (SK-) to yield an NBS gene clone (pNBS).

In addition, a DNA sequence encoding histidine at position 11 in an NBS polypeptide on pNBS was substituted with a sequence encoding alanine. Specifically, PCR with pNBS as the template was carried out using a primer 9 (5′-ggcCAGAGTTTGCCCGGAGAGC-3′; small letters denote a mutated region) shown in SEQ ID NO: 15 in the sequence listing and a primer 10 (5′-GCCGACCACTCTCTCCAG-3′) designed to anneal in a reverse direction to an adjacent region and shown in SEQ ID NO: 16 in the sequence listing. The primers used at that time were previously phosphorylated with T4 polynucleotide kinase. A reaction composition solution and reaction conditions are as follows.

Reaction composition solution (per 20 μL)

| | | |
|---|---|---|
| 5× iProof buffer | 4 | μL |
| 10 mM dNTP | 1.6 | μL |
| Phosphorylated primer 9 (10 pmol/μL) | 1 | μL |
| Phosphorylated primer 10 (10 pmol/μL) | 1 | μL |
| pNBS (1 ng/μL) | 1 | μL |
| iProof high-fidelity DNA polymerase (BioRad) | 0.2 | μL |
| H₂O | 11.2 | μL |

PCR condition
98° C. for 30 seconds×1 cycle
(98° C. for 30 seconds, 55° C. for 30 seconds and 72° C. for 55 seconds)×30 cycles
72° C. for 10 minutes×1 cycle
Then, cooled to 4° C.

A resulting fragment amplified by PCR was purified and self-ligation was performed by a ligation reaction to yield a vector containing the gene having the introduced mutation. The vector was introduced into Escherichia coli DH5α strain, and a plasmid was extracted from the resulting colony.

The same manipulation as the above was repeated in the resulting plasmid to substitute the DNA sequences encoding histidine at positions 14 and 67 in the NBS polypeptide with the sequence encoding alanine. At that time, in the substitution at position 14, a primer 11 (5′-ggcGTCGGCAgcCAGAGTTTGCC-3′; the small letters denote the mutated region) shown in SEQ ID NO: 17 in the sequence listing and a primer 12 (5′-TCTCTCCAGGCGGGCGCC-3′) designed to anneal in the reverse direction to the adjacent region and shown in SEQ ID NO: 18 in the sequence listing were used. In the substitution at position 67, a primer 13 (5′-ggcGTCGTAGATAACGAGGTTCCCG-3′; the small letters denote the mutated region) shown in SEQ ID NO: 19 in the sequence listing and a primer 14 (5′-AACAACAACGACGTGTGGGGG-3′) designed to anneal in the reverse direction to the adjacent region and shown in SEQ ID NO: 20 in the sequence listing were used.

As described above, a plasmid (pNBSH11AH14AH67A) containing the gene encoding the NBS variant was obtained.

### Example 3

An expression vector for the NAS variant (hereinafter referred to as pgFa3GNaHA1SJ) and an expression vector for the NBS variant (hereinafter referred to as pgFa3GNbHA1Ta) were constructed, and the dimeric protein of the present invention was obtained using the protein expression system using Aspergillus oryzae. The protein expression system has been designed to increase an amount of the secreted intended protein by using a-amylase in the host as a carrier protein. The system has been designed so that a KEX2 protease cleavage sequence (lysine-arginine) is inserted in a joint portion of α-amylase and the intended protein for the purpose of cutting out the carrier protein and further so that three glycine residues are inserted immediately before aspartic acid at N termini of the NAS variant and the NBS variant in order to further increase a cleavage efficiency. Details thereof have been disclosed in Example 11 in WO 2005/073372 previously filed by the applicants. In the regard, however, the genes inserted in the expression vectors were the NAS variant gene and the NBS variant gene.

Aspergillus oryzae NS-tApE strain (niaD-, sC-, tppA-, pepE-) was used as the host. The fungal strain is the strain obtained by disrupting a tppA gene and a pepE gene which are two protease genes in NS4 strain used in Example 11 in WO 2005/073372. It has been described that the amount of the expressed protein is increased by disrupting the protease gene. The fungal strain was given by Laboratory of Microbiology, Department of Biotechnology, Graduate School of Agricultural and Life Science, The University of Tokyo. Details for the fungal strain have been described in Seibutsu Kogaku Kaishi, 2006, Vol. 84, p. 351, and “Production of calf chymosin using the double protease-gene disruptant from A. oryzae” in Proceedings of the 58th Annual Meeting of the Society for Biotechnology, Japan, page 131, 1Hl1-4.

Hereinafter, the production of the expression vector is described.

By using a vector construction kit (Multisite Gateway Three-Fragment Vector Construction Kit; manufactured by Invitrogen), expression vectors (pgFa3GNaHA1SJ and pgFa3GNbHA1Ta) were prepared.

The method using the kit carries out the construction of expression vectors in the order of (1) preparation of 5′ entry clone, (2) preparation of the entry clone of an intended gene, (3) preparation of 3′ entry clone, and (4) preparation of an expression vector via the recombination between the three types of entry clones and a destination vector.

Specifically, the method was carried by the following procedures under the following conditions.

(1) Preparation of 5′ Entry Clone

A 5′-entry clone (hereinafter referred to as pg5′ PFa) containing amyB promoter and an ORF sequence was prepared. Details thereof have been disclosed in Example 11 in WO 2005/073372.

(2) Preparation of Entry Clone of Intended gene

The entry clone (hereinafter referred to as pE3GNaHA1) of the gene encoding the NAS variant among the intended genes was prepared as follows. Specifically, using the pNASH14AH36A as template, PCR was done using primer 15 comprising the nucleotide sequence shown in SEQ ID NO: 21 in the sequence listing as designed by adding the KEX2-cleavage sequence and three glycine residues before the N terminus (5′-GGGGACAAGTTTGTACAAAAAAGCAGGCTCTAAACGTGGGGGGGGGGACAGTGTCCTG CTCTCC-3′; the sequence of the attB site is underlined) and primer 16 comprising the nucleotide sequence shown in SEQ ID NO: 22 in the sequence listing as designed by adding a nucleotide sequence including a termination codon after the C terminus (5′-GGGGACCACTTTGTACAAGAAAGCTGGGTTTAATTAAGACTGCGGCACCC-3′; the sequence of the attB site is underlined).

An amplified fragment obtained by PCR was introduced into a donor vector pDONR221 (manufactured by Invitrogen), to obtain the entry clone pE3GNaHA1 of the gene encoding the NAS variant. Subsequently, pE3GNaHA1 was introduced into Escherichia coli DH5α strain, and a plasmid was extracted from the resulting colony.

The entry clone of the gene encoding the NBS variant (hereinafter referred to as pE3GNbHA1) was prepared essentially in the same manner as in the case of the gene encoding the NAS variant. Specifically, using the pNBSH11AH14AH67A as template, PCR was done using primer 17 comprising the nucleotide sequence shown in SEQ ID NO: 23 in the sequence listing as designed by adding the KEX2-cleavage sequence and three glycine residues before the N terminus (5′-GGGGACAAGTTTGTACAAAAAAGCAGGCTCTAAACGTGGGGGGGGGGACAGTGTCCTG CTCTCCG-3′; the sequence of the attB site is underlined) and primer 18 comprising the nucleotide sequence shown in SEQ ID NO: 24 in the sequence listing as designed by adding a nucleotide sequence including a termination codon after the C terminus (5′-GGGGACCACTTTGTACAAGAAAGCTGGGTTTATCCACCATTAACACGGCG-3′; the sequence of the attB site is underlined).

An amplified fragment obtained by PCR was introduced into a donor vector pDONR221 (manufactured by Invitrogen), to obtain the entry clone pE3GNbHA1 of the gene encoding the NBS variant. Subsequently, pE3GNbHA1 was introduced into Escherichia coli DH5α strain, and a plasmid was extracted from the resulting colony.

(3) Preparation of 3′ Entry Clone

A 3′ entry clone containing the AmyB terminator and an ATP sulfurylase (sC) gene from Aspergillus nidulans (hereinafter referred to pg3'sCJ) and a 3′ entry clone containing the AmyB terminator alone (hereinafter referred to as pg3′Ta) were prepared. Details thereof have been disclosed in Example 11 in WO 2005/073372.

(4) Preparation of Expression Vector via the Recombination Between Three Types of Entry Clones and Destination Vector

The three types of entry clones, namely pg5′ PFa obtained above in (1), pE3GNaHA1 obtained above in (2) and pg3'sCJ obtained above in (3) were mixed with a destination vector pDEST R4-R3 (manufactured by Invitrogen), to which LR Clonase Plus Enzyme Mix (manufactured by Invitroge) was added to promote the recombination between the attL site and the attR site whereby obtaining the intended NAS variant expression vector (pgFa3GNaHA1SJ). The preparation process and structure of such pgFa3GNaHA1SJ are shown in FIG. 3.

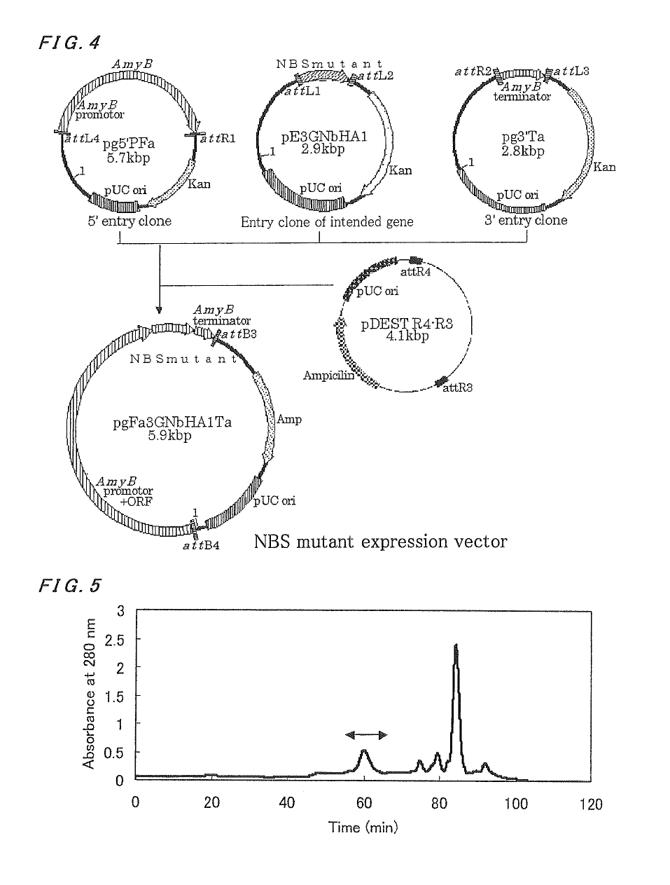

The three types of entry clones, namely pg5′ PFa obtained above in (1), pE3GNbHA1 obtained above in (2) and pg3′ Ta obtained above in (3) were mixed with a destination vector pDEST R4-R3 (manufactured by Invitrogen), to which LR Clonase Plus Enzyme Mix (manufactured by Invitrogen) was added to promote the recombination between the attL site and the attR site whereby obtaining the intended NBS variant expression vector (pgFa3GNbHA1Ta). The preparation process and structure of such pgFa3GNbHA1Ta are shown in FIG. 4.

It should be noted that details of the above techniques have been disclosed in Example 11 in WO 2005/073372.

(5) Transformation and Screening of Strain Having High-Productivity

Transformation was carried out in the following manner by a modification of the method of Punt et al. (“Methods Enzymol.”, Vol. 216, p. 447-457, 1992).

(a) Preparation of Protoplast

On a PD plate (39 g of potato dextrose (manufactured by Nissui) was dissolved in 1 liter of water and then autoclaved, and was then divided in a sterile plate), Aspergillus oryzae NS-tApE strain (niaD-, sC-, tppA-, and pepE-) was spread with a sterile bamboo skewer and incubated at 30° C. for 7 days, to grow a conidiospore.

The resultant was scratched into 100 ml of DPY culture broth (2% dextrin, 1% polypeptone, 0.5% yeast extract, 0.5% KH₂PO₄, 0.05% MgSO₄.7H₂O, pH 5.5) using a bamboo skewer for shaking culturing at 30° C. and 200 rpm for 20 hours. Subsequently, the bacterial cells were recovered with a sterile Miracloth (manufactured by Calbiochem) and rinsed in sterile water.

The cells were transferred into an L-shape test tube placing 10 ml of Sol-1 (1% Yatalase (manufactured by Takara Brewery), 0.6 M (NH₄)₂SO₄, 50 mM maleate buffer, pH 5.5), for gentle shaking at 30° C. for 3 hours, to prepare a protoplast.

(b) Introduction of Expression Vector

The resulting protoplast was passed through Miracloth to remove the cell debris, and an equal volume of Sol-2 (1.2 M sorbitol, 50 mM CaCl₂, 35 mM NaCl, 10 mM Tris-HCl, pH 7.5) was added. The resulting mixture was centrifuged at 2,000 rpm and 4° C. under break off.

After the precipitate was rinsed twice with Sol-2, the precipitate was suspended in 200 μl of Sol-2 to have a concentration of 1×10⁷ cell/ml.

2 μg of pgFa3GNaHA1SJ and 10 μg of pgFa3GNbHA1Ta were added, and the resulting mixture was incubated on ice for 30 minutes. 250 μl, 250 μl, and 850 μl of Sol-3 (60% PEG4000, 50 mM CaCl₂, 10 mM Tris-HCl, pH 7.5) was added in a step-wise manner, the mixture was gently mixed together, and then left to stand still at room temperature for 20 minutes. 5 to 10 ml of Sol-2 were added to the mixture followed by centrifuging and the precipitate was suspended in 500 μl of Sol-2.

The suspension was added to 5 ml of Top agar (supplemented with 1.2 M sorbitol) preliminarily divided and kept warm, and then poured onto the lower layer medium (MS plate; 1.2 M sorbitol, 0.2% NH₄Cl, 0.1% (NH₄)₂SO₄, 0.05% KCl, 0.05% NaCl, 0.1% KH₂PO₄, 0.05% MgSO₄.7H₂O, 0.002% FeSO₄.7H₂O, 2% glucose, 1.5% agar, pH 5.5) .

Then, a parafilm was wrapped and an air hole was opened thereon, followed by incubation at 30° C. for 3 to 5 days. The resulting colony was sub-cultured in an M plate (0.2% NH₄Cl, 0.1% (NH₄)₂SO₄, 0.05% KCl, 0.05% NaCl, 0.1% KH₂PO₄, 0.05% MgSO₄.7H₂O, 0.002% FeSO₄.7H₂O, 2% glucose, 1.5% agar, pH 5.5) three times, to stabilize the character. 30 transformant strains per one plate were obtained.

(c) Generation of Heterogeneous Protein at Small Scale in DYP Culture Broth (pH 8.0) and Recovery of Liquid Culture

12 transformant strains of the obtained transformants were spread on said M plate, for incubation at 30° C. for 2 to 4 days. Conidiospore was scratched and recovered with a bamboo skewer sterilized in an autoclave, and cultured by shaking in a 100-ml flask charged with 20 ml of DPY culture broth (pH 8.0) (in the composition of the reagents in the DPY culture broth (pH 5.5) described in (a), 0.5% KH₂PO₄ was replaced with 0.5% K₂HPO₄ and the pH was adjusted to 8 with 1 M NaOH solution) at 30° C. and 200 rpm for 3 days. The cells were separated and recovered from the liquid culture with a Miracloth.

(d) Screening of Strain Having High-Productivity and Western Blotting Analysis

The recovered liquid culture was subjected to SDS-PAGE (15% acrylamide) under reducing conditions, transferred onto a PVDF film (manufactured by Millipore), and analyzed by western blotting.

The film after the transfer was soaked in TEST containing 5% skim milk, and shaken gently at room temperature for 60 minutes. 0.5 μl of an anti-neoculin antibody as a primary antibody was added to 2.5 ml of TBST containing 5% skim milk, and then placed in a plastic bag with the film and left to stand still therein at room temperature for 60 minutes. After the film was washed with TEST for 10 minutes three times, 2.5 μl of alkali phosphatase-bound anti-rabbit IgG antibody (manufactured by Sigma) as a secondary antibody was added to TBST containing 5% skim milk and was then placed in a plastic bag with the film, and was left to stand still at room temperature for 60 minutes. After the film was washed with TEST for 10 minutes three times, the film was transferred to a mixture obtained by adding 66 μl of NBT and 33 μl of BCIP as substrates to 10 ml of a reaction solution (0.1M Tris-HCl (pH 9.5), 5 mM MgCl₂, 0.1 M NaCl). The resulting mixture was reacted under a condition of light-shielding darkness for several minutes, for color reaction. From the results, the fungal strain which expressed the dimeric protein of the present invention at high level was selected, and designated as Aspergillus oryzae NStApE-NCLHA strain. The strain has been deposited as an accession No. FERM BP-10799 at the International Patent Organism Depositary, the National Institute of Advanced Industrial Science and Technology, Chuo-6, 1-1, Higashi 1-chome, Tsukuba-shi, Ibaraki-ken, Japan.

(6) Mass-Scale Culturing

(a) Recovery of Conidiospore

Conidiospore of Aspergillus oryzae NStApE-NCLHA strain was spread on a PD plate using a bamboo skewer, and incubated at 30° C. for 3 to 7 days. 10 ml of 0.01% Tween 80 solution was poured on the plate with the conidiospore growing thereon, to scratch and recover the conidiospore with a sterile dropping pipette (manufactured by Sarstedt)

This was recovered in a 15 ml Falcon, for vigorous agitation for one minute. Then, the cell debris was discarded with a sterilized Miracloth. After centrifugation at 4° C. and 4000 rpm for 5 minutes, the supernatant was discarded. 10 ml of 0.01% Tween 80 solution was added to the resulting precipitate, for agitation. After repeating the same procedure once again, the resulting solution was centrifuged at 4° C. and 4000 rpm for 5 minutes, to discard the supernatant. The precipitate was dissolved in 1 ml of sterile water. Several microliters of the recovered solution were diluted to about 10 fold with water, to count the conidiospore using a Thoma counter chamber.

(b) Mass-Scale Shaking Culture in DPY Culture Broth (pH 8.0)

120 ml each of the DPY culture broth (pH 8.0) was charged in 500 ml flask, and five sets thereof were prepared. The conidiospore was added to the individual flasks to have a concentration of 1×10⁷ cell/liter, for shaking culture at 30° C. and 200 rpm for 72 hours. About 400 ml of culture medium was recovered after cells were discarded with a Miracloth.

(7) Purification

(a) Ammonium Sulfate Fractionation

Ammonium sulfate was added to the culture medium recovered from the mass-scale culture described above in (6) so that a saturated concentration of ammonium sulfate was 60%. Then, the resulting mixture was incubated for 30 minutes. After centrifugation at 10000 rpm and 4° C. for 30 minutes, the resulting precipitate was recovered.

(b) Purification by Phenyl Hydrophobic Column Chromatography

The precipitate obtained by the ammonium sulfate fractionation was dissolved in buffer A (3 M NaCl, 20 mM acetate-Na, pH 5.0), and then dialyzed against the buffer A overnight. After recovery, the solution was passed through a 0.45 μm filter. The resulting filtrate was defined as sample.

As the column, HIC PH-814 (20×150 mm) (manufactured by Shodex) was used. Fractionation was done by phenyl hydrophobic column chromatography where the buffer A was used at the period for 0-20 minutes, a gradient from 0 to 100% of buffer B (20 mM acetate-Na, pH 5.0) was applied at the period for 20-90 minutes and the buffer B was used at the period for 90-110 minutes as a mobile phase, under a flow condition of 3.0 ml/min (detection; 280 nm). The result is shown in FIG. 5. In FIG. 5, a horizontal axis denotes an elution time and a vertical axis denotes an absorbance at 280 nm. Fractions around the elution time of 60 minutes (region shown by an arrow in FIG. 5) were recovered. Portions of the obtained fractions were electrophoresed, and western blotting analysis was carried out to identify the elution of the intended protein. These fractions were further purified.

(c) Purification by Gel Filtration Column Chromatography

The fractions obtained by purification by the aforementioned phenyl hydrophobic column chromatography was dialyzed against water, freeze-dried and dissolved in a small amount of water. The resulting solution was used a sample.

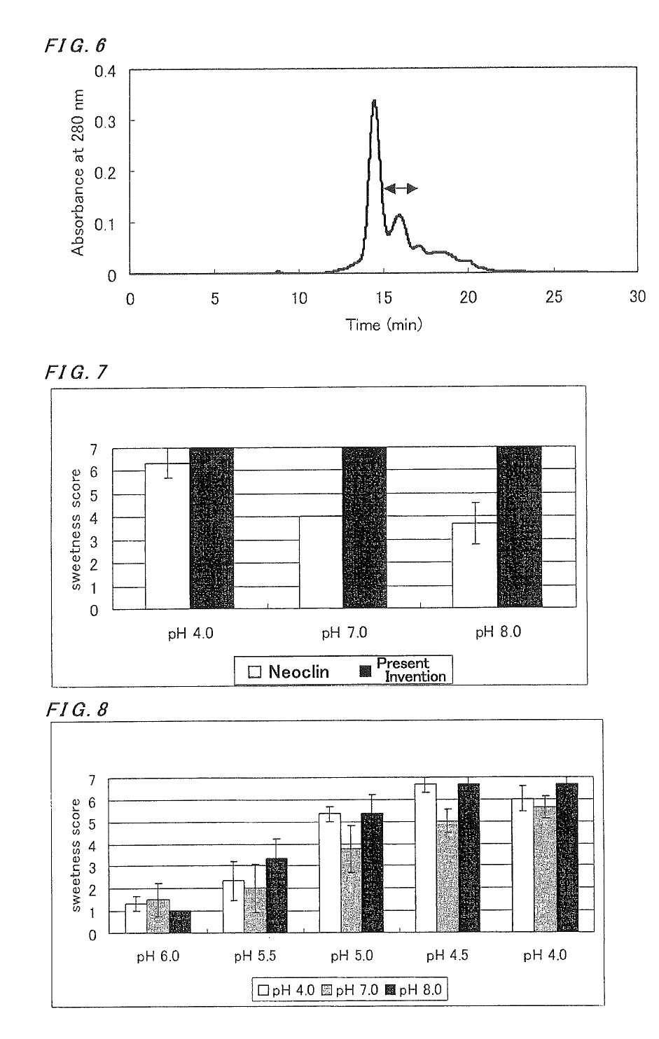

Using TSK-GEL G3000SW (7.5×300 mm) (manufactured by TOSOH) as the column, fractionation was done by gel filtration column chromatography using 0.5 M NaCl and 50 mM acetate-Na, pH 5.0 as the mobile phase, under a flow condition of 1.0 ml/min (detection; 280 nm). The result is shown in FIG. 6. In FIG. 6, the horizontal axis denotes the elution time and the vertical axis denotes the absorbance at 280 nm. Fractions around the elution time of 16 minutes (region shown by an arrow in FIG. 6) were recovered. Portions of the obtained fractions were electrophoresed, and western blotting analysis was carried out to identify the elution of the intended protein. These fractions were dialyzed against the water, and then freeze-dried, thereby completing the purification.

(8) Analysis of Sugar Chain

The sugar chain in the variant was analyzed according to the following procedure. That is, a sugar composition of the sugar chain was analyzed using the dimeric protein purified in (7) as the sample and using ABEE sugar composition analysis Kit Plus S (manufactured by Honen Corporation). Further, SDS-PAGE of the purified dimeric protein was also carried out to estimate a molecular weight of the NAS variant.

Specifically, using the purified dimeric protein as a sample, the sugar was converted to a reduced sugar. Continuously the reduced sugar was hydrolyzed with an acid, to cleave all the glycoside bonds contained in the sugar chain of the glycoprotein, whereby releasing the sugars in the forms of monosaccharides. After the generated monosaccharides were labeled and then separated by HPLC using TSKgel ODS-80TsQA column (manufactured by TOSOH; a 4.6 ram diameter×7.5 cm), detection was performed by 305 nm absorbance for analysis.

As a result of the analysis, the sugar composition of the sugar chain added to the NAS variant included mannose and N-acetylglucosamine at a ratio of 9:2. A trace amount of galactose was detected.

As a result of SDS-PAGE, the molecular weight of the NAS variant was about 14 kDa. Since the estimated molecular weight calculated from the amino acid sequence of the NAS variant was 12,253, it was predicted that about 2 kDa of the sugar chain was added to the NAS variant.

From the above, it was estimated that the NAS variant was glycosilated with the high mannose type N-linked sugar chain which was typical for filamentous fungi belonging to genus Aspergillus. Examples of the estimated sugar chain were shown in FIG. 2.

### Example 4

Sensory evaluation was performed, and the sweetness intensity of the dimeric protein of the present invention was evaluated at different pH's. The dimeric protein purified in Example 3 was used as the dimeric protein of the present invention. The neoculin obtained by purifying neoculin produced from Aspergillus oryzae NS-NAB2 strain which was a neoculin high production strain was used as control neoculin. As for the strain, the production method thereof, and the purification method thereof, there were employed the methods described in detail in Example 11 in WO 2005/073372.

The evaluation was performed by rating the sweetness with the sweetness scores of 1 to 7 depending on the sweetness intensity. The “sweetness score” used herein refers to the score obtained by comparing the sweetness intensity with the sweetness intensity of aspartame. Each score corresponds to the sweetness intensity of aspartame at each concentration as follows.

Score 7: >2.0 mM
Score 6: 2.0 mM
Score 5: 0.5 to 2.0 mM
Score 4: 0.5 mM
Score 3: 0.1 to 0.5 mM
Score 2: 0.1 mM
Score 1: <0.1 mM

That is, the scores 4, 6, and 7 mean that the sweetness intensity is 12.5 times, 50 times, and more than 50 times as strong as the sweetness intensity of aspartame in molar concentration ratio, respectively.

The sensory evaluation was carried out by four panelists. The panelist fasted for 30 minutes before the sensory test. The panelist first tasted 300 μL of 0.1, 0.5, and 2.0 mM aspartame solutions and remembered the sweetness intensity of each solution. Subsequently, the dimeric protein of the present invention or neoculin was dissolved at 40 μM (1.0 mg/ml) concentration in three kinds of solutions at different pH's (100 mM sodium acetate (pH 4.0), water (pH 7.0), and 100 mM ammonium acetate (pH 8.0)). 100 μL of each solution was put on panelist's tongue, adapted for 30 seconds, and then the sweetness thereof was evaluated.

The results are shown in FIG. 7. At that time, neoculin at pH 4.0 exhibited the sweetness corresponding to the 2.0 mM aspartame solution. The sweetness intensities of neoculin at pH 7.0 and pH 8.0 were reduced and corresponded to the sweetness of the 0.5 mM aspartame solution. Meanwhile, the dimeric protein of the present invention exhibited the sweetness equivalent to or stronger than the sweetness of the 2.0 mM aspartame solution regardless of pH. From the above results, it was confirmed that the dimeric protein of the present invention had the sweetness independent of pH.

### Example 5

After tasting the dimeric protein of the present invention or neoculin in the above sensory evaluation, the panelist tasted 300 μL of 100 mM sodium acetate solutions at different pH's (pH 4.0, 4.5,5.0,5.5, 6.0), and evaluated the intensity of the sweetness induced by the acid, i.e., a taste-modifying activity.

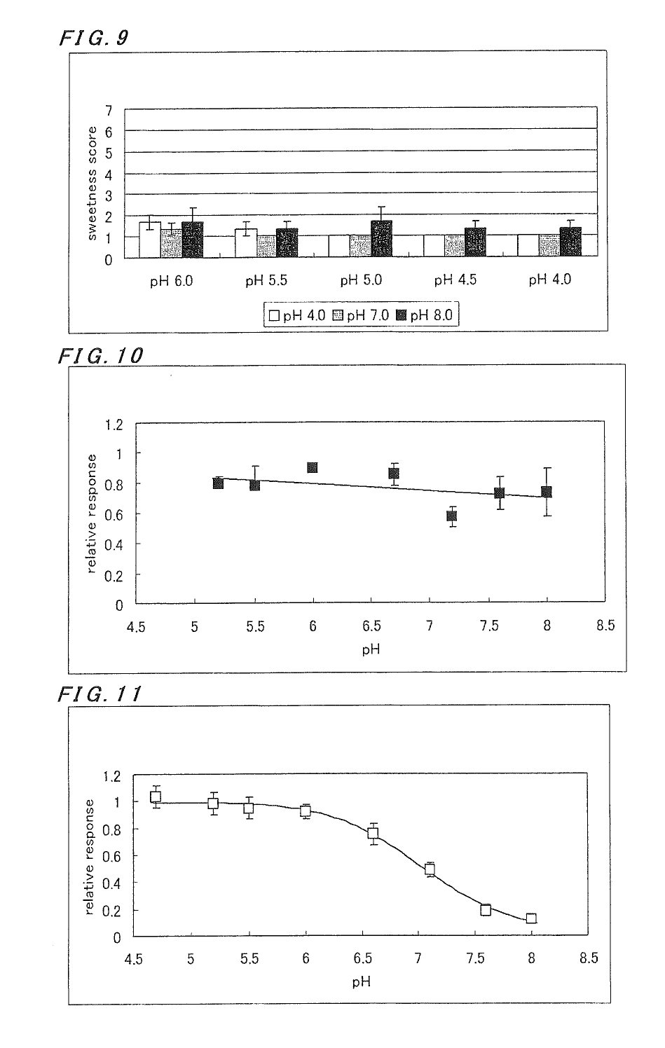

The sweetness intensity sensed when the sodium acetate solution at each pH was tasted was evaluated in the same way as in Example 4. The results by neoculin and the dimeric protein of the present invention are shown in FIGS. 8 and 9, respectively. In the figures, the horizontal axis denotes pH of the sodium acetate solution, and pH in a three-bar graph denotes pH of the solution in which the protein was dissolved in Example 4.

As a result, in neoculin, the sweetness was increased dependently as the pH was lowered (FIG. 8), whereas in the dimeric protein of the present invention, no taste-modifying activity like neoculin was observed after any solution was tasted (FIG. 9). From the above, it has been shown that the dimeric protein of the present invention has no taste-modifying activity that remains in the oral cavity for a long period of time as is observed in neoculin, and that the dimeric protein is excellent as the sweetener.

### Example 6

A signaling system was constructed by co-expressing hT1R2-hT1R3 which is a human sweetness receptor and a chimera Gα protein in cultured HEK293T cells. The activity of the dimeric protein of the present invention was evaluated using the cultured cell by a calcium imaging method.

That is, a sample to be evaluated was prepared as a tastant solution, and the cultured cells in which the signaling system had been constructed were stimulated with the tastant solution. The cells responded at that time were detected by monitoring intracellular calcium concentrations to evaluate a response intensity. By using the evaluation system, it is possible to objectively evaluate data for the sweetness intensity of the sample to be evaluated. Details thereof are described below.

The evaluation system was developed by the inventors of the present invention, and presented in the Annual Meeting of the Japan Society for Bioscience, Biotechnology and Agrochemistry, in 2007.

The plasmid in which cDNA of hT1R2 shown in SEQ ID NO: 25 in the sequence listing and cDNA of hT1R3 shown in SEQ ID NO: 26 in the sequence listing had been introduced in pEAK10 vector (manufactured by Edge Biosystem) was given by Dr. Charles Zuker (University California of San Diego) and Dr. Nicholas J. P. Ryba (NIDCD).

A gene encoding G15Gi3 (which is constructed by replacing C terminal 5 amino acid residues of G15 which acts upon various G protein-coupled receptors with those of Gi3, and is known to efficiently act upon the sweetness receptor) which is the chimera Gα protein comprising the amino acid sequence shown in SEQ ID NO: 27 in the sequence listing was produced by PCR and introduced into pcDNA3.1(+) (manufactured by Invitrogen).

That is, the gene was amplified by PCR with the plasmid including full length of rat G15 gene sequence (accession number: AB015308) as the template, using a primer 19 (5′-AAAAGGCGCGCCGCCATGGCCCGGTCCCTGACT-3′) shown in SEQ ID NO: 30 in the sequence listing and a primer 20 (5′-AAAAGCGGCCGCTCAGTAAAGCCCACATTCGTCCAGGTACCGGGCCAGCA-3′) shown in SEQ ID NO: 31 in the sequence listing, and using KOD DNA polymerase (TOYOBO).

The primer 19 is a forward primer having the structure of 4 adenine bases-AscI restriction site-Kozak sequence-5′ terminal 18 bases of rat G15 gene. The primer 20 is a reverse primer having the structure of 4 adenine bases-NotI restriction site-terminal codon-15 bases which replace a partial amino acid sequence to ECGLY-20 bases of rat G15 gene.

A PCR product treated and cut out with AscI-NotI was introduced in a product obtained by cutting pEAK10 vector with AscI-NotI to produce the G15Gi3 gene. It should be noted that the chimera Gα protein was published in Proc. Natl. Acad. Sci. USA, 99:4692-4696, 2002.

The tastant solution was prepared by adding a tastant, citric acid, and NaOH to assay buffer (10 mM 2-[4-(2-hydroxyethyl)-1-piperazinyl]ethanesulfonic acid (HEPES), 130 mM NaCl, 10 mM glucose, 5 mM KCl, 2 mM CaCl₂, and 1.2 mM MgCl₂, pH 7.2) so that the final pH after the addition to the cells was adjusted to 4.7 to 8.0. As the tastant, the dimeric protein (5.0 μM) of the present invention purified in Example 3, neoculin (20.0 μM) for the purpose of a control experiment, or aspartame (10 mM) for the purpose of obtaining the relative intensity was selected.

It should be noted that, as described below, when the cells are stimulated with the tastant solution, the concentration of the tastant is diluted to 1.5 times with the solution previously placed in a container of the measurement system.

HEK293T cells were seeded in a 100 mm dish (manufactured by Corning) and cultured in Dulbecco's modified Eagle's medium (manufactured by Sigma) supplemented with 10% fetal bovine serum (manufactured by JRH) and 0.05% penicillin-streptomycin (Sigma-aldrich) under the condition at 37° C. and 5% CO₂. Subculture was performed every three days before being confluent.

For transfection, one day before transfection was performed the cells were seeded onto 35 mm diameter dish so that the cells reached 80 to 90% confluency on the following day. The vector containing cDNA of hT1R2, the vector containing cDNA of hT1R3, the vector containing the gene encoding the chimera Gα protein, and pDsRed2-N1 (Takara Bio) as a marker for transfection efficiency were introduced into HEK293T cells using Lipofectamine 2000 reagent (Invitrogen) so that the weight ratio was 4:4:1:0.2 and introduced amounts were 4.6 to 5.5 μg as a DNA introduction efficiency. A transfection efficiency was about 60 to 80% as estimated from the fluorescence of DsRed2.

The medium for the cells was changed 6 hours after the transfection, and 50% of the cells were subcultured. These cells were detached by treating with trypsin 24 hours after the transfection, and 10⁶ cells were seeded in a 96-well plate (Nunc) having a black side wall. The medium was recovered 48 hours after the transfection, and 100 μL of a solution prepared by dissolving 2.5 μM calcium ion indicator Fura2-AM (Molecular Probe) in the above assay buffer was loaded thereto, and the plate was incubated at 37° C. for 30 minutes. Subsequently, the cell was rinsed with the above assay buffer and 50 μl of the solution was left in the well. Then the plate was incubated at room temperature for 20 minutes. Subsequently, 100 μL of the tastant solution produced above (the tastant was dissolved in the assay buffer) was added to the well using a pipette to stimulate the cells.

The change of the intracellular calcium concentration was represented as a ratio of fluorescence intensity (F340/F380) emitted at two excitation wavelengths of 340 nm and 380 nm calculated by measuring the fluorescence at absorption wavelength of 510 nm. Filter changer (Lambda 10-2, Sutter), MicroMax cooled CCD camera (Princeton Instrument), and an inverted fluorescence microscope (IX70, Olympus), all of which are controlled by a computer, were used for the measurement.

It should be noted that images of the measurement results were recorded over 90 seconds after the stimulation of the cells. In order to prevent a non-specific response of the cells due to the stimulation with acid, the measurement was completed within 60 minutes after being left to stand still at room temperature.

The measurement results are shown in FIGS. 10 and 11. In the figures, the horizontal axis denotes pH of the tastant solution. A “relative response” in the vertical axis denotes a value calculated by normalizing the number of responding cells to the taste stimulation relative to the number of the responding cells to the aspartame solution at high concentration (6.7 mM). It should be noted that the cell was defined to be “responding cell” when the increase in the F340/F380 ratio between 30 and 90 seconds after the taste stimulation was more than 0.15. The cells stimulated with the solution containing no tastants scarcely responded.

As a result, the response of the cells to the dimeric protein of the present invention did not attenuate in any region of the acidic region, the neutral region, and the basic region (FIG. 10). Meanwhile, the response of the cells to neoculin, which was performed as the control experiment, was attenuated in the neutral and basic regions where pH was 6 or higher. For example, the response was reduced to about a half around pH 7 which was the neutral region, and attenuated to about a tenth around pH 8 which was the basic region (FIG. 11). From these, it was shown that the dimeric protein of the present invention had the sweetness independent of pH, differently from neoculin.

### INDUSTRIAL APPLICABILITY

According to the present invention, the novel polypeptide NAS variant and polypeptide NBS variant, which are different from the known NAS and NBS polypeptides and have the novel amino acid sequences are provided, and the novel dimeric protein per se having sweetness independent of pH can be provided using these polypeptides.

Further, the novel composition which can be applied to foods and has sweetness independent of pH can be provided using the dimeric protein.

What is claimed is:

1. Gene encoding a neoculin basic subunit (NBS) variant comprising a polypeptide having substitution mutation or deletion mutation in one or several amino acids of a NBS polypeptide, wherein the NBS variant has sweetness independent of pH when the NBS variant is combined with a polypeptide comprising an amino acid sequence shown in SEQ ID NO: 28.

2. Recombinant vector comprising the gene according to claim 1.

3. A transformant carrying the recombinant vector according to claim 2.

4. The gene encoding the NBS variant according to claim 1, wherein the NBS variant comprises the amino acid sequence of SEQ ID NO: 2, having said substitution mutation or said deletion mutation.

5. The gene encoding the NBS variant according to claim 4, wherein said NBS variant, comprising the amino acid sequence of SEQ ID NO: 2, has an amino acid substitution at position 11 of SEQ ID NO: 2.

6. The gene encoding the NBS variant according to claim 5, wherein said NBS variant further includes substitution at at least one of position 14 and position 67 of SEQ ID NO: 2.

7. The gene encoding the NBS variant according to claim 6, wherein said NBS variant includes substitution at both position 14 and position 67 of SEQ ID NO: 2.
